(19) United States
(12) Patent Application Publication
(10) Pub. No.: US 2024/0081852 A1
(43) Pub. Date: Mar. 14, 2024

(54) SOFT TISSUE CUTTING DEVICE WITH VISUALIZATION AND/OR PRESSURE SENSING

(71) Applicant: Sonex Health, Inc., EAGAN, MN (US)

(72) Inventors: DARRYL E. BARNES, EAGAN, MN (US); JAY SMITH, BYRON, MN (US); DAVID HERRIDGE, MENDOTA HEIGHTS, MN (US)

(73) Assignee: Sonex Health, Inc., EAGAN, MN (US)

(21) Appl. No.: 18/367,765

(22) Filed: Sep. 13, 2023

Related U.S. Application Data
(60) Provisional application No. 63/406,623, filed on Sep. 14, 2022.
(60) Provisional application No. 63/426,182, filed on Nov. 17, 2022.

(51) Int. Cl. A61B 17/32

(52) U.S. Cl. CPC A61B 17/320036; A61B 2017/320048; A61B 2217/002

(57) ABSTRACT

A method of performing a carpal tunnel release procedure on a hand may involve advancing a shaft of a soft tissue cutting device into the hand, positioning a distal end of the shaft at or near a carpal tunnel, sensing a first pressure in the hand, using a pressure sensor of the soft tissue cutting device, moving a blade along the shaft to cut a transverse carpal ligament, and sensing a second pressure in the hand, using the pressure sensor.

### CROSS REFERENCE TO RELATED APPLICATIONS

The present application claims the benefit of and priority to U.S. Prov. Pat. App. No. 63/406,623, filed Sep. 14, 2022 and to U.S. Prov. Pat. App. No. 63/426,182, filed Nov. 17, 2022, the disclosures of which are incorporated herein by reference.

### TECHNICAL FIELD

The present disclosure relates to medical devices and methods. More specifically, the present disclosure relates to devices and methods for cutting soft tissue with visualization and/or pressure sensing capabilities.

### BACKGROUND

Carpal tunnel syndrome affects approximately 3.7% of the general population and up to 7% of manual labor workers. Some cases of carpal tunnel syndrome are treated with a surgical carpal tunnel release procedure (“CTR procedure”). The standard CTR procedure involves forming an incision on the palm side of the hand, near the wrist, then finding and cutting the transverse carpal ligament to reduce median nerve compression and carpal tunnel pressures in the carpal tunnel region. Although the standard CTR procedure is highly effective, it can result in relatively long recovery times, due to the difficulty of fully healing the incision on a portion of the hand that moves frequently (i.e., the palm). Thus, less invasive CTR devices and methods have been developed.

Examples of less invasive devices used for CTR surgery are described in U.S. Pat. Nos. 10,357,272, 11,259,829, and 10,864,055, the full disclosures of which are hereby incorporated by reference. In some embodiments, the devices described in the above-referenced patents are used with external ultrasound visualization of the carpal tunnel region and the hand and/or wrist, to find and cut the transverse carpal ligament while avoiding nearby nerves and blood vessels. Although this external visualization with ultrasound works quite well, it may also be advantageous to have different or additional visualization and/or sensing options, to allow a physician to even better understand where the target tissue is, where nearby nerves and blood vessels are located, whether the target tissue has been cut, etc.

### BRIEF SUMMARY

This disclosure describes a device and method for cutting soft tissue in the hand and/or wrist. The primary embodiments are described for use in cutting the transverse carpal ligament to treat carpal tunnel syndrome, but in alternative embodiments the devices and methods described herein may be used to cut soft tissue in other parts of the body. For example, other nerve impingement syndromes in the hand, elbow, shoulder, or other joint(s) may be treated using the devices and methods described in this disclosure. In embodiments described herein, the devices and methods include a visualization component, a pressure sensor, an alternative sensor for sensing one or more parameters in the patient's body, or some combination thereof. External ultrasound may also be used in any of the embodiments described herein.

In one aspect of the present disclosure, a method of performing a carpal tunnel release procedure on a hand may involve advancing a shaft of a soft tissue cutting device into the hand, positioning a distal end of the shaft at or near a carpal tunnel, using a visualization member on the distal end of the shaft to visualize at least one anatomical landmark, moving a blade along the shaft to cut a transverse carpal ligament, and removing the shaft of the soft tissue cutting device from the hand. According to various embodiments, the visualization member may include, but is not limited to, a camera, a light fiber, and an ultrasound transducer.

In some embodiments, the method may further include sensing pressure within the carpal tunnel using a pressure sensor in the soft tissue cutting device. For example, sensing pressure may involve sensing a first pressure before cutting the transverse carpal ligament and sensing a second pressure after cutting the transverse carpal ligament. The method may also involve converting the first pressure and the second pressure into digital numerical data and providing the digital numerical data on a display coupled with the soft tissue cutting device. In some embodiments, sensing pressure involves sensing pressure in at least one balloon coupled with the shaft of the soft tissue cutting device, and the pressure sensor is located in a handle of the soft tissue cutting device.

The method of claim 1, further comprising inflating at least one balloon coupled with the shaft of the soft tissue cutting device to move one or more soft tissues away from the blade of the soft tissue cutting device.

In another aspect of the disclosure, a method of performing a carpal tunnel release procedure on a hand may include advancing a shaft of a soft tissue cutting device into the hand, positioning a distal end of the shaft at or near a carpal tunnel, sensing a first pressure in the hand using a pressure sensor of the soft tissue cutting device, moving a blade along the shaft to cut a transverse carpal ligament, and sensing a second pressure in the hand, using the pressure sensor. Optionally, the method may further include visualizing an area within the hand using a visualization member on the distal end of the shaft. In some embodiments, the method may also include inflating at least one balloon coupled with the shaft of the soft tissue cutting device to move one or more soft tissues away from the blade of the soft tissue cutting device. Sensing the first pressure and the second pressure may involve sensing the first pressure and the second pressure in the at least one balloon, with the pressure sensor being located in a handle of the soft tissue cutting device.

In yet another aspect of the disclosure, a device for performing a carpal tunnel release procedure on a hand may involve a handle comprising a slider, a shaft extending from the handle, a blade on a distal portion of the shaft, coupled with the slider, and a visualization member on the distal portion of the shaft. The visualization member may be a camera, a light fiber, an ultrasound transducer, or any other suitable visualization component. In some embodiments, the device may also include at least one balloon coupled with the distal portion of the shaft and configured to move one or more soft tissues away from the blade. Such embodiments may further include a pressure sensor in the handle that senses pressure in the at least one balloon. For example, the pressure sensor may include, but is not limited to, one or more pressure transducers or thermocouples.

In another aspect, a device for performing a carpal tunnel release procedure on a hand may include a handle with a slider, a shaft extending from the handle, a blade on a distal portion of the shaft, coupled with the slider, at least one balloon coupled with the shaft, and a pressure sensor in the handle and coupled with the at least one balloon to sense pressure in the at least one balloon. Optionally, the device may also include a visualization member coupled with the distal portion of the shaft, such as but not limited to a camera, a light fiber, or an ultrasound transducer.

In some embodiments of the device, the shaft has an upper surface with an opening, and the blade moves out of the opening to cut tissue. The device may also include a ramp in the shaft along which the blade rides as it moves back and forth in the shaft. The ramp is configured to move the blade out of the opening in the shaft to cut tissue. In some embodiments, the device includes two balloons disposed on two sides of the shaft, configured to move soft tissue away from the blade.

Additional aspects and embodiments are described in greater detail below, in connection with the attached drawing figures.

### BRIEF DESCRIPTION OF DRAWINGS

FIG. 1 is an upper perspective view of a tray for a device according to a non-limiting illustrative embodiment.

FIG. 2 is a lower perspective view of FIG. 1.

FIG. 3 is an exploded perspective view of the tray of FIG. 1 shown without a device.

FIG. 4 is a top view of the tray of FIG. 1 showing various positions of a device with the tray.

FIG. 5 is a perspective view of a soft tissue cutting device with a portion of its outer housing removed to illustrate interior detail.

FIG. 6 is partial perspective view of a blade and blade activation assembly of the soft tissue cutting device shown in FIG. 5.

FIG. 7 is a perspective view of the device shown in FIG. 5 with its outer housing removed to illustrate interior detail.

FIG. 8 is a side perspective view of the tray of FIG. 1 with the device housed in the housing slot.

FIG. 9 is a top view of the tray of FIG. 1 with the device housed in the priming slot.

FIG. 10 is an upper perspective view of the tray of FIG. 1 with the device near the safety slot when the priming has been correctly performed.

FIG. 11 is an upper perspective view of the tray of FIG. 1 with the device received in the safety slot when the priming has been incorrectly performed.

FIG. 12 is an enlarged perspective view of the safety slot of FIG. 10.

FIG. 13 is an enlarged perspective view of the safety slot of FIG. 11.

FIG. 14 is a top view of a soft tissue cutting device with visualization and sensing capabilities, according to one embodiment.

FIGS. 15A and 15B are perspective and top views, respectively, of a soft tissue cutting device with pressure sensing, according to one embodiment.

### DETAILED DESCRIPTION

The following detailed description is illustrative in nature and is not intended to limit the scope, applicability, or configuration of the disclosure in any way. Rather, the following description provides some practical illustrations for implementing examples of the present disclosure.

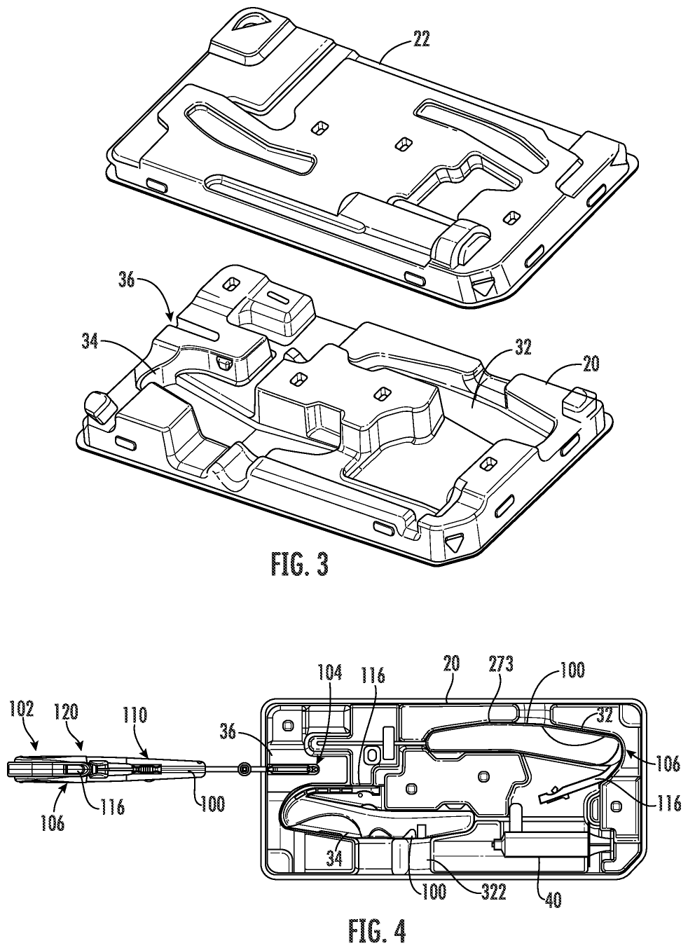

FIGS. 1-13 generally illustrate a soft tissue cutting device and a tray for housing and helping prime the device. While the illustrated embodiments show an illustrative embodiment of a soft tissue cutting device, such as a transverse carpal ligament cutting device, many different types of devices can be shipped, primed, and tested prior to use according to systems and methods disclosed herein. In various embodiments, the soft tissue cutting device illustrated in FIGS. 4-13 may include a visualization component, a pressure sensing component, a different sensing component, or any combination thereof.

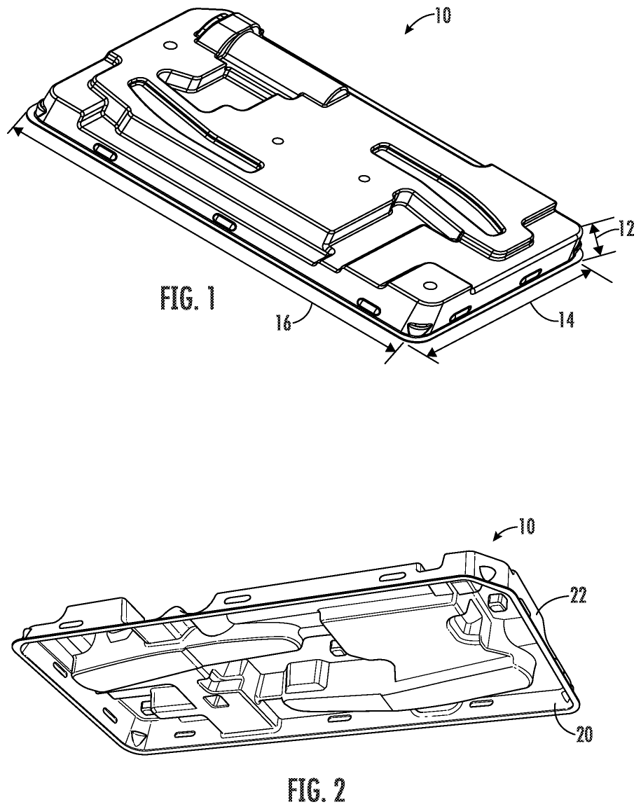

FIGS. 1 and 2 are upper and lower perspective views of a tray 10 for enclosing a device 100 (not shown) according to an illustrative embodiment. Tray 10 has a tray thickness 12, a tray width 14, and a tray length 16. Tray 10 includes a body 20 and a cover 22.

The tray 10 can be made by molding polymers such as resins or copolyesters. In one embodiment, the tray 10 is made by molding polyethylene terephthalate-glycol (PET-G). Other materials are within the scope of the present disclosure. In addition, in certain embodiments, the tray 10 may be shipped in a sealed bag (not shown) so as to ensure that the tray 10 and the device (e.g., device 100) housed therein are sterile.

In some embodiments, the tray 10 has generally rounded corners and may not have sharp edges. Advantageously, this may promote ease of use during surgery. For instance, the rounded edges and corners of the tray 10 prevent catching on and/or tearing of surgical gloves worn by medical personnel when grasping and/or manipulating the tray 10. In the illustrated embodiment, the tray 10 is generally rectangular in shape, with a tray thickness 12 less than tray width 14 and tray length 16, though other shapes are contemplated within the scope of the present disclosure.

In certain illustrative embodiments, the tray length 16 is between about 10 inches and about 20 inches. Further, in some embodiments, the tray width 14 is between about 5 inches and about 10 inches, and the tray thickness 12 is between about 0.5 inch and 3 inches. In an illustrative embodiment, the tray length 16 is about 16 inches, the tray width 14 is about 7.5 inches and the tray thickness 12 is about 1.33 inches. These dimensions are not limiting.

FIG. 3 is an exploded perspective view of the tray 10 according to an illustrative embodiment. The tray 10 includes a body 20, a cover 22, and a plurality of slots 32, 34, and 36. The cover 22 can be removably connectable to the body 20. When the cover 22 is connected to the body 20 of the tray 10, the cover 22 substantially encloses the body 20 and its contents. In certain advantageous embodiments, the cover 22 can be a sterile cover, and may not be removed except when the tray 10 is in use for a procedure, such as a CTR procedure.

FIG. 4 is a perspective view of the tray 10 with a device 100 shown in various positions within the tray 10. The tray includes a body 20 and a plurality of slots 32, 34, 36. Each of the slots 32, 34, and 36 of the tray 10 receive and/or are connected to the device 100 to illustrate various positions of the device 100 when housed in different slots 32, 34, 36 of the tray 10. In certain embodiments, the tray 10 is shipped with a single device 100 housed in the slot 32 and a priming syringe 40 as will be described further below.

In certain embodiments, the device 100 as illustrated in FIG. 4 can be a soft tissue cutting device 100, such as described in the commonly-assigned application Ser. No. 14/854,775 (“the '775 application”), the entire contents of which is hereby incorporated by reference. The present disclosure is not limited to the disclosed embodiment of device 100 in the '775 application and many variants thereof are contemplated within the scope of the present disclosure.

With continued reference to FIG. 4, the device 100, according to an embodiment, can be a soft tissue cutting device, such as a transverse carpal ligament cutting device. The device 100 has a proximal end 102 and a distal end 104. The proximal end 102 includes a handle 106 and a balloon activation lever 116. The handle 106 includes a blade activation assembly 110. The device 100 also includes an inflation assembly 120 coupled to the handle 106. The inflation assembly 120 and blade activation assembly 110 each control various functions of the shaft at the distal end 104.

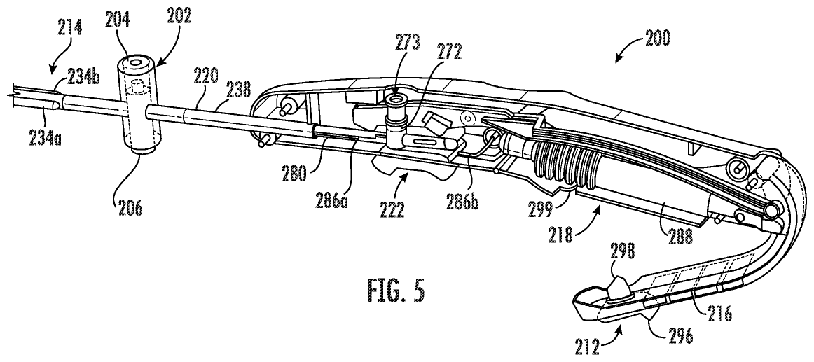

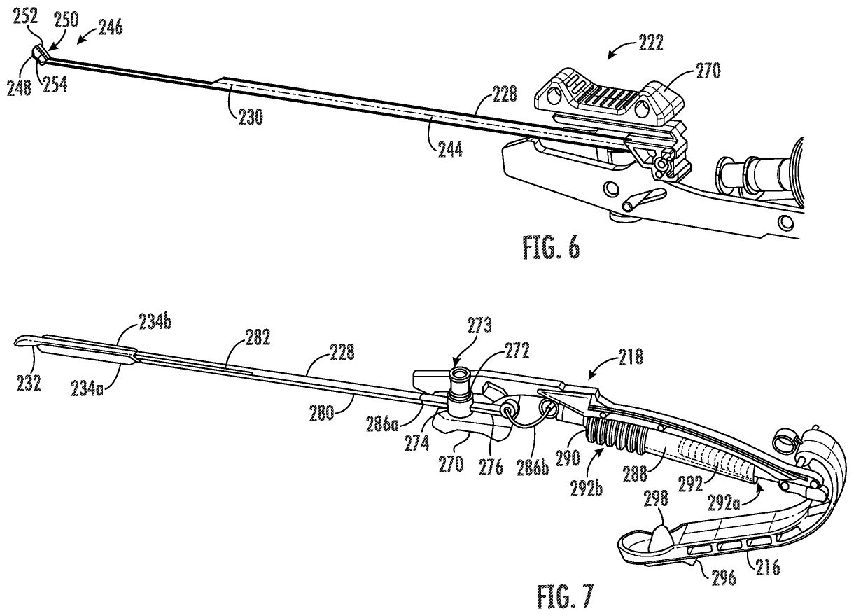

FIGS. 5, 6 and 7 are perspective views of a soft tissue cutting device 200 according to an embodiment of this disclosure. FIG. 5 is a perspective view of the soft tissue cutting device 200 with its outer housing removed to illustrate interior detail. FIG. 6 is partial perspective view of a blade activation assembly 222 and a blade 228 of the soft tissue cutting device 200 shown in FIG. 5. FIG. 7 is a perspective view of the device 200 shown in FIG. 5 with its outer housing removed to illustrate interior detail.

The device 200 can be substantially similar to device 100. The device 200 has a proximal end 212 and distal end 214. The proximal end 212 includes a balloon activation lever 216 (similar to the balloon activation lever 116 of device 100). The distal end 214 includes an axial depth indicator 202 with ends 204 and 206. The axial depth indicator is located on a shaft 220 that has a top surface 238. A blade 228 is housed at least partially within the shaft 220. A pair of balloons 234a and 234b is located on shaft 220. Device 200 also includes an inflation assembly 218 and a blade activation assembly 222.

The shaft 220 of device 200 can have a tip that is smooth edged (e.g., rounded) to permit ease of insertion into the carpal tunnel region. The shaft 220 can optionally be echogenic and/or include diagnostic equipment to permit visualization of the carpal tunnel region during insertion or surgery. The axial depth indicator 202 can be a spring-loaded pin that is slidable along the shaft 220 by pressing ends 204 and 206 toward each other. The axial depth indicator 202 can thus be used to indicate the depth of insertion of the distal end 214 into the patient.

The balloons 234a and 234b expand radially outwardly from the shaft 220. When inflated, the balloons 234a, 234b have a spherical, oval, bilobular or other configuration. When deflated, the balloons 234a, 234b are generally flush with the shaft 220. The balloons 234a and 234b can be positioned anywhere along the shaft 220 such that they, for example, expand (or create) the safe zone in a patient's carpal tunnel region. While two lateral balloons 234a, 234b are illustrated, any number of radially expanding balloons can be used and be placed anywhere about the shaft 220 to secure the distal end 214 in position within the carpal tunnel region and to expand the safe zone. For instance, in an illustrative embodiment, as the radially expanding balloons 234a, 234b inflate, they move the flexor tendons, median nerve, and ulnar neurovascular bundle away from the device 200 to help create a region in which the transverse carpal ligament can be cut while preventing nearby at-risk structures from being cut, effectively increasing the “safe zone.”

The balloons 234a, 234b can have any desired configuration that allows them to inflate and deflate. In some embodiments, the entire balloon is expandable and thus inflates and deflates. In other embodiments, only part of the balloon is expandable. For example, the balloon can have a fixed portion and an expandable portion. The balloons 234a, 234b also inflate to a desired size selected to accommodate the size of a patient's wrist (and thus the patient's carpal tunnel region). For example, in some cases, the balloons 234a, 234b can be provided with a specific size such that when they are fully inflated, they have a specific inflated size. In one embodiment, each of the balloons 234a, 234b inflate to a similar or same diameter (e.g., a diameter of about 1.5 mm). In patients with larger wrists, larger balloons can be used. In patients with smaller wrists, smaller balloons can be used. In another embodiment one of the balloons 234a, 234b inflates to one size and another inflates to a different size. In other cases, the balloons 234a, 234b can have a standard size but can be partially inflated or fully inflated to have a variety of different inflated sizes. In some cases, the balloon inflation can be graded to allow the operator to choose a particular balloon diameter. In certain cases, the balloon inflation can be pressure dependent, such that the balloon manually or automatically inflates until a specific pressure is exerted on the balloon surface.

The blade activation assembly 222 actuates the blade 228 through a slot 232 located on distal end 214. In the illustrated example, the slot 232 is positioned near (and between) the balloons 234a, 234b, though, of course, the slot 232 may be positioned at other locations in the distal end 214. The blade activation assembly 222 is coupled to a blade shaft 244. The blade 228 includes a blade working end 246. The blade shaft 244 has a longitudinal axis 230 along which the blade 228 is actuated. In the illustrated embodiment, the blade working end 246 is configured as a hook and configured to cut a transverse carpal ligament. The blade working end 246 includes an inner surface 250 that adjoins an outer surface 248 at a point 252. The blade working end 246 also includes a cutting edge 254. In some embodiments, the cutting edge 254 can be non-parallel to the blade shaft 244. Alternatively, the cutting edge 254 can be non-linear (e.g., curved). The blade shaft 244 engages with the blade activation assembly 222 to move the blade 228 forward (distally) and backward (toward the proximal end 212) in a direction parallel to the longitudinal axis 230.

In some embodiments, instead of including a cutting edge 254, at least part of the entire outer surface 248 (or substantially the entire outer surface 248 or the entire outer surface 248) can be a dull and/or blunt surface that would not cut body tissue. Likewise, the point 252 may be a dull and/or blunt surface. Additionally, a limiting stop can be positioned near the blade working end 246 to engage with and/or travel within a guided surface (e.g., a groove, guide, track or tunnel) that guides the forward and rearward movement of the blade 228. Such embodiments are advantageous, as they assist in cutting the target tissue in a controlled fashion, while preventing inadvertent cutting of tissue other than the target tissue.

The blade activation assembly 222 includes a slider button 270 positioned on an external surface of the balloon activation lever 216. The slider button 270 can move in both forward and backward directions (parallel to the longitudinal axis 230) to move the blade 228 forward and backward. An operator can engage the slider button 270 with a finger (for example by engaging a thumb with the slider button 270). When the slider button 270 is moved to extend the blade 228, the blade working end 246 protrudes through the slot 232 in the distal end 214.

The inflation assembly 218 includes a fluid coupling 272 and a fill port 273. In the embodiment shown, the fluid coupling 272 is T-shaped and includes a first port 274 and a second port 276 and is connected to the fill port 273. The inflation assembly 218 also includes a first tube 280, a second tube 282, a first conduit 286a, a second conduit 286b, and an inflation device 288.

The fluid coupling 272 is coupled to the fill port 273. The first port 274 is fluidly coupled to (e.g., by fluid fittings such as push-fit or barbed fitting) a first conduit 286a. The second port 276 is fluidly coupled to a second conduit 286b (e.g., by fluid fittings such as push-fit or barbed fitting). The first conduit 286a is fluidly coupled to the first tube 280 and the second tube 282. The first tube 280 is fluidly coupled to the first balloon 234a, and the second tube 282 is fluidly coupled to the second balloon 234b.

Accordingly, when the fill port 273 engages with a priming device (e.g., priming syringe) that supplies pressurized fluid (e.g., by a plunger), the fluid travels through the fluid coupling 272, into the first conduit 286a (via the first port 274), through the first tube 280 and the second tube 282 and into the first balloon 234a and the second balloon 234b to inflate and pressurize the balloons.

The second conduit 286b is fluidly coupled to the inflation device 288. In the embodiment shown, the inflation device 288 is located inside of the balloon activation lever 216, though in other embodiments, the inflation device 288 can be located outside of the balloon activation lever 216. The inflation assembly 218 described in this embodiment can also be included in any of the other device embodiments (e.g., device 100) described herein. In the embodiment shown, the inflation device 288 is a bellows 288 that includes a body 290 and a spring-biased plunger 292 with a distal end 292a and a proximal end 292b. The body 290 is shown as a transparent feature to illustrate interior detail. The spring-biased plunger 292 is pivotally coupled to the balloon activation lever 216. When the balloon activation lever 216 is depressed, the plunger 292 moves towards distal end 292a. When the balloon activation lever 216 is released, the spring-bias of the plunger 292 moves the plunger 292 toward proximal end 292b.

In other embodiments, the inflation device 288 can be in the form of a syringe with a piston or plunger or the like. Alternatively, the inflation device 288 can be any desired device known in the art that holds inflation material and both pushes inflation material out of the inflation device 288 and pulls inflation material back into the inflation device 288. The inflation device 288 is actuated by the balloon activation lever 216 to inflate or deflate the balloons 234a, 234b once primed.

When the balloons 234a, 234b are deflated, the fluid from the balloons 234a, 234b travel to and stay within the bellows 288. In order to re-inflate the balloons 234a, 234b, the balloon activation lever 216 is engaged, thereby applying pressure on the distal end 292a of the plunger 292 of the bellows 288 to pressurize fluid within the bellows 288. This causes fluid to move into the first conduit 286a, through the first tube 280 and the second tube 282, and into the balloons 234a, 234b. When the operator desires to deflate the balloons 234a, 234b, the balloon activation lever 216 is released. The plunger 292 of the bellows 288 then moves toward the proximal end 292b as a result of spring bias, thereby pulling fluid back towards the inflation device 288. As a result, the fluid moves out of the balloons 234a, 234b and back into first tube 280 and second tube 282, through the first conduit 286a, and into the bellows 288.

The balloon activation lever 216 includes a balloon locking mechanism. In the embodiment shown, the balloon locking mechanism includes a spring-biased button 296, which terminates in a pin 298. The device 200 includes a groove 299 for receiving the pin 298 of the button 296. If the operator desires to lock the balloons 234a, 234b in an inflated position, the balloon activation lever 216 is depressed to bring the spring-biased button 296 toward the groove 299 (e.g., by an operator's thumb or finger). The pin 298 of the spring-biased button 296 is then received within the groove 299. When the pin 298 rests within the groove 299, the plunger 292 of the inflation assembly 218 is held in tension against its spring-bias, thereby maintaining the position of the balloon activation lever 216 in the depressed position and the plunger 292 near the proximal end 292b of the bellows 288. As a result, the fluid would continue to be pressurized, thereby maintaining the balloons 234a, 234b in an inflated position.

Prior to use, the device 200 is in a first inactive position. In the first inactive position, the blade 228 positioned such that the cutting edge 254 is fully housed and protected within the shaft 220 and the device 200 can be safely inserted into a carpal tunnel region. Additionally, the balloons 234a, 234b are in a deflated configuration. The operator inserts the distal end 214 of the device 200 into a carpal tunnel region such that the transverse carpal ligament is positioned adjacent the top surface 238 of the shaft 220. The operator then engages the balloon activation lever 216 to cause the balloons 234a, 234b inflate and expand the safe zone within the carpal tunnel region. The balloons 234a, 234b can then be locked in the inflated state using the balloon locking mechanism to engage the spring-biased button 296 to maintain the pin 298 in the groove 299.

The operator next moves the slider button 270 to move the blade working end 246 to protrude out of the shaft 220, and fully expose the cutting edge 254 to cut the transverse carpal ligament. The operator continues to move the slider button 270 to move the blade working end 246 (and cutting edge 254) in a desired direction (e.g., backward). As the cutting edge 254 moves along the desired direction (e.g., backward), it cuts the transverse carpal ligament. Once the transverse carpal ligament is cut, the operator releases the spring-biased button 296 and the balloon activation lever 216. As a result of the spring-bias, the plunger 292 moves toward distal end 292a. As a result, the balloons 234a, 234b deflate. In this second position, the blade 228 is positioned such that the blade working end 246 does not protrude out of the shaft 220, the cutting edge 254 is fully housed and protected within the shaft 220, and the device 200 can thus be safely removed from the body.

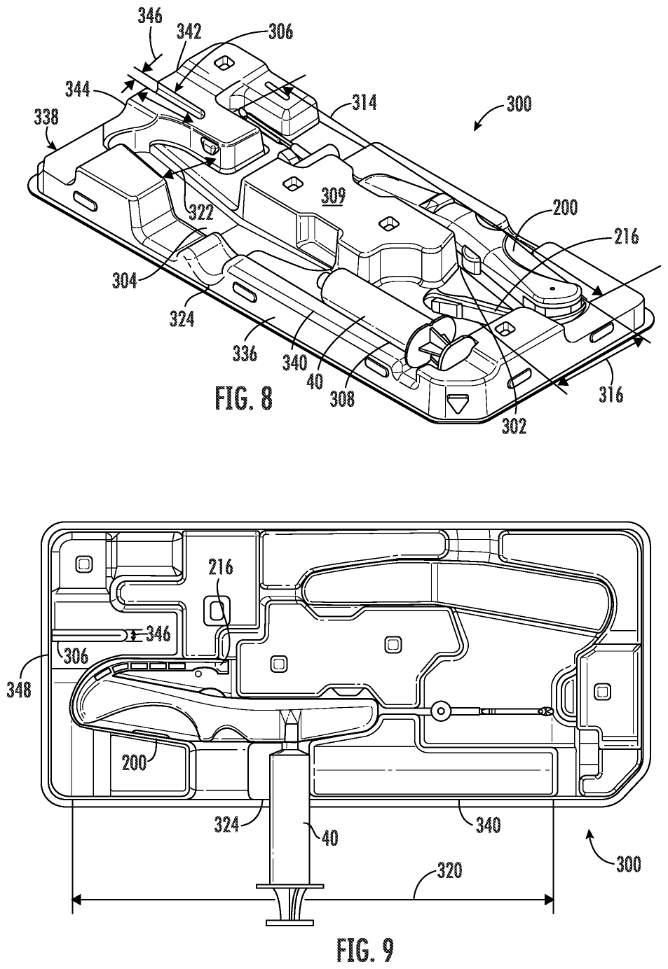

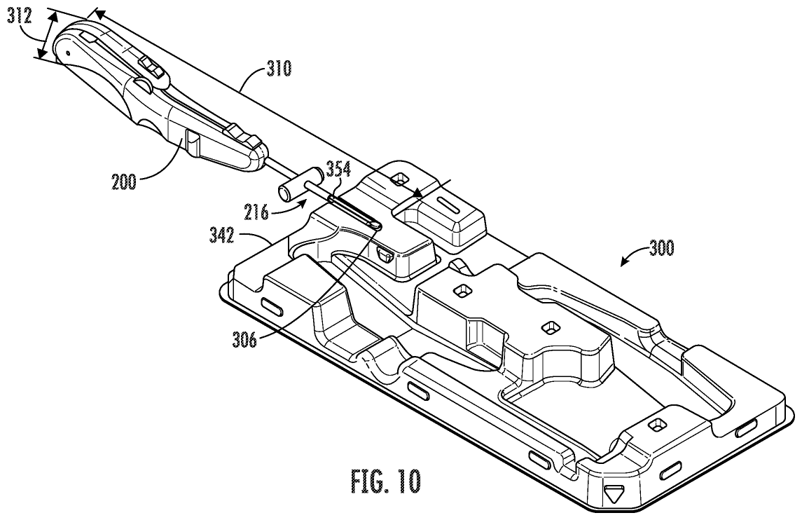

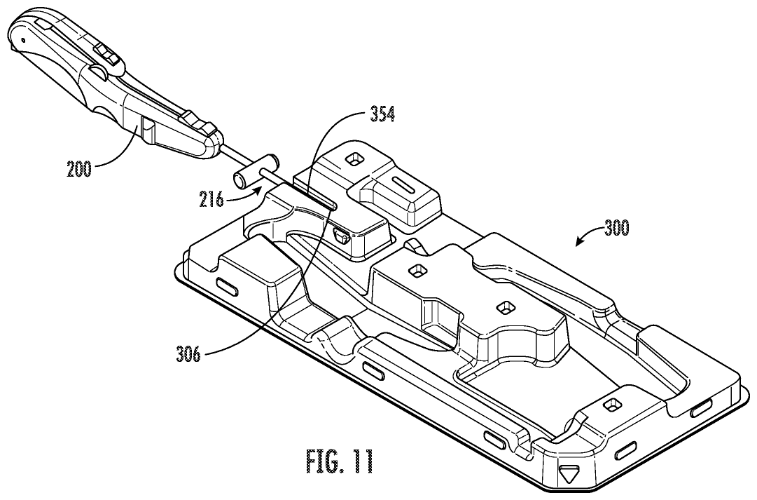

FIG. 8 is a side perspective view of a tray 300 with the device 200 in a housing slot 302 and the priming syringe 40 in a priming syringe slot 308. FIG. 9 is a top view of the tray 300 with the device 200 housed in a priming slot 304. FIG. 10 is an upper perspective view of the tray 300 with the device 200 resting on safety slot 306 when priming of the device 200 has been correctly performed. FIG. 11 is an upper perspective view of the tray 300 with the device 200 received in the safety slot 306 when priming of the device 200 has not been correctly performed.

Referring to FIGS. 8-11, the tray 300 includes the housing slot 302, the priming slot 304, the safety slot 306, the priming syringe slot 308, and a top surface 309. The device 200 has a device length 310 and a device width 312. The housing slot 302 has a housing slot length 314 and a housing slot width 316. The priming slot 304 has a priming slot length 320 and a priming slot width 322. A lateral groove 324 is adjacent to the priming slot 304. The tray 300 also includes a first side surface 336 and a second side surface 338, both of which are perpendicular to the top surface 309. A first edge 340 adjoins the top surface 309 and the first side surface 336. A second edge 342 adjoins the top surface 309 and the second side surface 338. The safety slot 306 has a safety slot length 344 and a safety slot width 346.

As shown in FIG. 8, the priming syringe 40 can be supplied along with the device 200 and housed in the tray 300 in the form of a surgical kit. During shipment and prior to use, the device 200 can be removably housed in the housing slot 302. Further, the priming syringe 40 can be removably housed in the priming syringe slot 308. The housing slot 302 is elongated in shape. As shown in FIG. 8, the elongated shape of the housing slot 302 generally matches the elongated shape of the device 200. The housing slot 302 is a cavity recessed from the top surface 309 of the tray 300. The priming syringe slot 308 is also a cavity recessed from the top surface 309 and is shaped to match the shape of the priming syringe 40.

The housing slot 302 can be shaped to match the shape of the device 200 prior to priming to receive the device 30 in a snug fashion. For instance, the housing slot width 316 can be equal to (or slightly greater than) the device width 312 and the housing slot length 314 can be equal to (or slightly greater than) the device length 310. The housing slot 302 is shaped such that the balloon activation lever 216 of the device 200 is in an extended (or released) position. The priming slot 304 is recessed from the top surface 309 and can be shaped to generally match the shape of the device 200 during a priming procedure. The priming slot length 320 can be about equal to the housing slot length 314. In some embodiments, the priming slot width 322 can be slightly less than the housing slot width 316, because, as discussed below, during priming, the balloon activation lever 216 is in a position different from its position when in the housing slot 302.

The safety slot 306 is located on the second edge 342 adjoining the top surface 309 and the second side surface 338. The safety slot 306 is recessed from the top surface 309 and is generally elongated in shape. The safety slot length 344 is generally shorter than the housing slot length 314 and/or the priming slot length 320. Further, in some embodiments, the safety slot width 346 can be narrower than both the housing slot width 316 and the priming slot width 322.

As shown in FIG. 9, once the device 200 is ready for priming, the device 200 can be removed from the housing slot 302, and the priming syringe 40 can be removed from the priming syringe slot 308 and placed laterally near the fill port 273. Once removed, the device 200 can be positioned and aligned with the lateral groove 324 so as to facilitate connection to the priming syringe 40. The lateral groove 324 is defined on the first side surface 336 near the first edge 340. As shown in FIG. 9, during priming, a portion of the priming syringe 40 is positioned and held within the lateral groove 324, such that the distal tip of the priming syringe 40 can engage with the fill port 273 of the device 200 positioned in the priming slot 304. When the device 200 is positioned in the priming slot 304, the balloon activation lever 216 is in an intermediate position between the depressed position and the extended position. The balloon activation lever 216 is movable to the depressed position to inflate the balloons after priming, and to the extended position to deflate the balloons after priming.

In order to prime the device 200 prior to use, in certain illustrative embodiments, the priming syringe 40 is used. In one example, priming involves filling the balloons with a fluid and initially pressurizing the balloons prior to use. For example, the priming syringe 40 can have a plunger which is used to dispense a fluid through the distal tip into a fluid conduit and thereby into the balloons of the device 200. The plunger can be movable with respect to the body of the priming syringe 40. Other types of priming devices (e.g., injectors, pumps, and the like) are also contemplated within the scope of the present disclosure.

In some embodiments, the priming syringe 40 can be removably connectable with the fill port on the device 200. For instance, the removable connection between the fill port and the priming syringe 40 can be a Luer-Lok® connection. The fill port in turn can be fluidly connected to the balloons by way of conduits. Thus, the priming syringe 40 can be fluidly coupled to the balloons such that movement of the plunger of the priming syringe 40 can create a vacuum and/or deflate the balloons, or fill the balloons with a predetermined volume of fluid and/or inflate the balloons. In one embodiment, when the plunger of the priming syringe 40 is drawn, a vacuum is created within the balloons. When the plunger of the priming syringe 40 is depressed, the balloons may be filled with a predetermined volume of fluid. Further, the balloons can be deflated and re-inflated successively to equalize balloon pressure to the pressure within the syringe, as will be described further below.

In an illustrative embodiment, the priming syringe 40 contains about 20 milliliters of saline, and pressurizes the balloons by providing between about 1 milliliter and about 2 milliliters of saline. However, the volumes provided herein should not be construed as limiting. Further, fluids other than saline can also be used to prime the balloons.

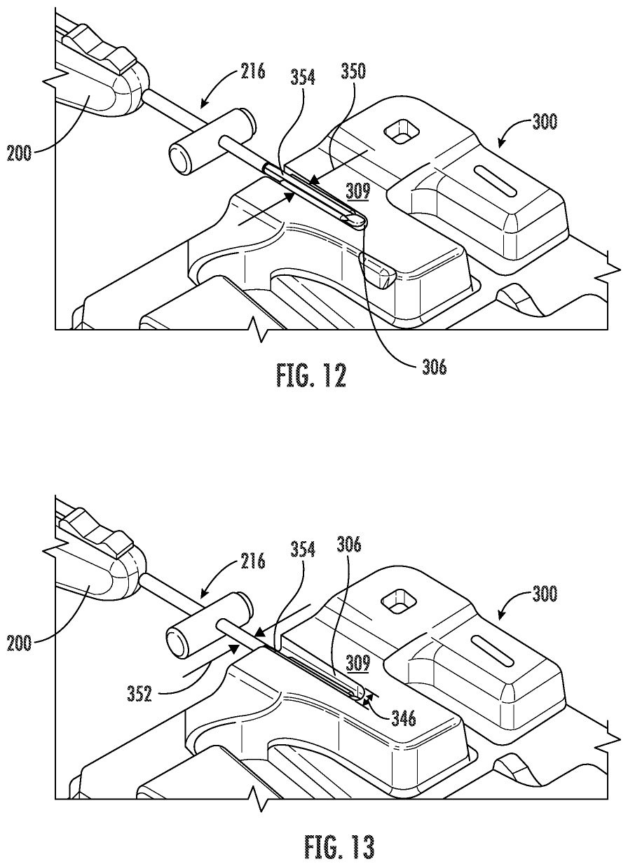

After the device 200 has been primed, the safety slot 306 is used to determine whether the device 200 is primed such that it is ready for use. FIGS. 10-13 illustrate a test for determining whether the balloons have been properly primed for use. FIG. 12 is an enlarged perspective view of the device 200 in safety slot 306 as shown in FIG. 10. FIG. 13 is an enlarged perspective view of the device 200 in safety slot 306 as shown in FIG. 11.

FIGS. 10 and 12 show the device 200 in the safety slot 306 when the device 200 is properly primed. When the device 200 is properly primed, the balloons are inflated such that the distal end of the device 200 has a first distal end width 350. When the device 200 is properly primed, the first distal end width 350 is larger than the safety slot width 346 such that the distal end of the device 200 does not fall into in the safety slot 306 and instead rests on top of the safety slot 306. Furthermore, a top portion 354 of the distal end extends above the top surface 309 of the tray 300. This indicates that the balloons are sufficiently inflated for use and thus that the device 200 has been properly primed.

FIGS. 11 and 13 show the device 200 in the safety slot 306 when the device 200 is not properly primed. When the device 200 has not been properly primed, the balloons are underinflated such that the distal end of the device has a second distal end width 352 that is narrower than the first distal end width 350. When the balloons have not been properly primed, the second distal end width 352 is narrow enough such that the distal end of the device 200 falls into the safety slot 306 and is received within the safety slot 306. Furthermore, the top portion 354 of the distal end does not protrude past the top surface 309 of the tray 300. This indicates that the balloons are not sufficiently inflated for use and that the device 200 has not been properly primed.

Embodiments of the present disclosure include a method of priming the device 200. Referring to FIGS. 8-13, the method includes providing the device 200 according to any of the disclosed embodiments (or equivalents thereof) enclosed in the tray 300. The device 200 is enclosed in the tray 300 such that the device 200 is positioned in the housing slot 302 such that the balloon activation lever 216 is in the extended position. The priming syringe 40 is positioned in the priming syringe slot 308.

The device 200 is then removed from the housing slot 302 and the balloon activation lever 216 is depressed into an intermediate position such that the device 200 can be positioned in the priming slot 304. The balloon activation lever 216 is only depressed sufficiently to place the device 200 into the priming slot 304 and is not fully depressed. The priming syringe 40 is then removed from the priming syringe slot 308 and filled with a volume of a fluid (e.g., sterile saline). In one embodiment, the volume of fluid is 20 milliliters (ml). The priming syringe 40 is then placed into lateral groove 324 and attached to the fill port of the device 200.

To prime the device 200, the plunger of the priming syringe 40 is depressed a first time to deliver a priming volume of the fluid through the fill port and into the balloons to fill the balloons with the fluid. In one embodiment, the priming volume of fluid is between about 1 ml and 2 ml. The plunger is then drawn back to create a vacuum and thereby deflate the balloons. In certain embodiments, after the plunger is drawn back, the vacuum can be held for a first-time interval. In an illustrative embodiment, the first-time interval is between about 1 second and about 10 seconds (e.g., 5 seconds).

After the plunger is drawn back to create a vacuum, the plunger is then depressed a second time to refill the balloon with a priming volume of the fluid. In one embodiment, the priming volume of fluid is between about 1 ml and 2 ml. The plunger is then again drawn back to create a vacuum and thereby deflate the balloons a second time. In certain embodiments, after the plunger is drawn back, the vacuum can be held for a second time interval. In an illustrative embodiment, the second time interval is between about 1 second and about 10 seconds (e.g., 5 seconds).

After the plunger is drawn back to create a vacuum for a second time, the plunger is then depressed a third time to refill the balloons with the second volume of the fluid. In one embodiment, the second volume of fluid is between about 1 ml and 2 ml. In certain embodiments, the plunger may or not be drawn back and/or depressed again after the plunger has been depressed a third time. Additionally, in some embodiments, after the plunger is depressed a third time, the plunger may be held in its position for a third time interval. In an illustrative embodiment, the third time interval can be between about 1 second and about 5 seconds (e.g., 2 seconds). The plunger is then released after being held during the third time interval and the pressure within the balloons subsequently equalizes with the pressure within the priming syringe 40.

After the plunger is depressed a third time and the pressure in the balloons is equalized, the plunger is not drawn back and the priming syringe 40 is disconnected from the fill port of the device 200. The device 200 is then be removed from the priming slot 304, thereby permitting the balloon activation lever 216 to automatically un-depress and move to the extended position as a result of its spring-biasing.

After removal of the device 200 from the priming slot 304, the balloon activation lever 216 can be depressed to activate (inflate) the balloons. When the balloons are in an activated state, the cutting blade of device 200 becomes active and can be tested by gently moving the slide button on the handle of the device to expose the cutting knife from its distal recessed position. The slide button is used to cycle the cutting blade a minimum of one time from its distal to proximal recessed positions to ensure functionality.

With the balloon activation lever 216 remaining depressed and the balloons activated, a safety test can now be performed to determine whether the tissue cutting device 200 has been properly primed. The distal end of the tissue cutting device 200 is positioned near the safety slot 306. The axial depth indicator 202 can be optionally rotated so as to be non-parallel (e.g., perpendicular) to the balloon activation lever 216. Advantageously, this step permits the axial depth indicator 202 from interfering with the safety test, for instance, by abutting any surface and thereby inadvertently causing small axial extensions of the distal end of the device 200. The distal end of the device can then be gently placed in the safety slot 306. In some embodiments, pressure should not be applied on the distal end of the device 200 so as to force the distal end into the safety slot; the distal end 104 should simply be positioned over the safety slot 306.

When the device 200 is properly primed, the balloons are inflated such that the distal end of the device 200 has a first distal end width 350. When the device 200 is properly primed, the first distal end width 350 is larger than the safety slot width 346 such that the distal end of the device 200 does not fall into in the safety slot 306 and instead rests on top of the safety slot 306. It may also be visually apparent to the operator that the balloons are properly inflated. Furthermore, a top portion 354 of the distal end should extend above the top surface 309 of the tray 300. This indicates that the balloons are sufficiently inflated for use and thus that the device has been properly primed. If the balloons are not properly primed and are underinflated such that the distal end of the device 200 falls into the safety slot 306 and is received within the safety slot 306, the device 200 should be primed again and the safety test should be performed again until the device 200 passes the test.

Once the device 200 has been primed and has passed the safety test, the device 200 can be used to perform a procedure, such as cutting a transverse carpal ligament to treat carpal tunnel syndrome. To prepare the device 200 for such a procedure, the balloon activation lever 216 is released into an extended position such that the balloons are deflated. The device 200 can then be placed within the carpal tunnel of the patient, and its placement with respect to different anatomical features (e.g., median nerve, ulnar vessels, superficial palmar arterial arch, palmar digital nerves, and the transverse carpal ligament, etc.) can be verified. The balloons 234a, 234b are then inflated by depressing the balloon activation lever 216, which simultaneously unlocks the cutting blade. The balloons are then locked in the inflated position used the spring-biased button 296.

The cutting blade is then be deployed by using the slider button 270. For instance, the blade can be slid in a direction from the distal end of the device 200 toward the proximal end. Accordingly, the cutting blade would engage and cut a distal transverse carpal ligament from the distal end to the proximal end of device 200. After cutting, the slider button 270 is slid distally so as to place the blade in its distal resting position. The spring-biased button 296 is then released and the balloons are deflated by releasing the balloon activation lever 116 to the extended position. The device 200 is then removed from the patient and post-surgical care (e.g., wound closure, applying bandages, etc.) is administered.

In any of the embodiments described above, a soft tissue cutting device (such as but not limited to the device 200) may include one or more visualization components, one or more sensors for sensing a parameter (or parameters) in the body, or any suitable combination thereof. One embodiment of such a soft tissue cutting device is described in further detail immediately below, but any of the features described below may also be applied to (or included in) any of the embodiments described above.

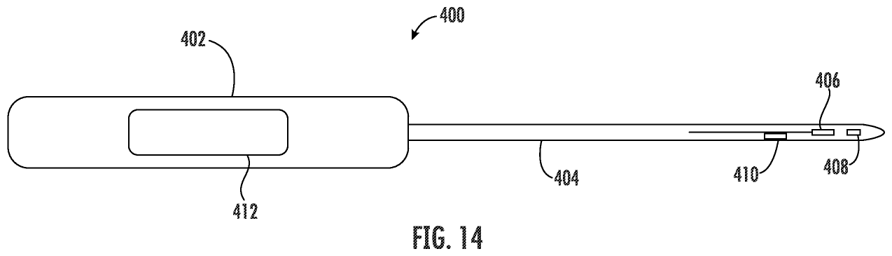

Referring now to FIG. 14, another embodiment of a soft tissue cutting device 400 is illustrated. In this embodiment, the soft tissue cutting device 400 includes a handle 402, a shaft 404, a moveable blade 406, a camera 408 (e.g., a visualization member), a pressure sensor 410, and a video screen 412 on the handle 402. Alternative embodiments may include only the camera 408, only the pressure sensor 410, or alternative visualization and/or sensing components. In various embodiments, the camera 408 may be any type of small camera using any suitable type of image capturing technology, such as but not limited to CMOS, CCD, camera on a chip, fiber optics, or the like. In other embodiments, other visualization technologies (e.g., other visualization members) may be used instead of the camera 408, such as but not limited to one or more light fibers to transilluminate tissues, ultrasound transducer(s), or the like. In various embodiments, the camera 408 may send video signals back to the handle 402 wirelessly or via electronic wiring extending through the shaft 404. Alternatively, video signals may be sent wirelessly (e.g., Bluetooth, WiFi (e.g., GE Vscan Air)) or via wired connection from the camera 408 to a video monitor (not shown), which may be connected to the soft tissue cutting device 400. Such a video monitor may be any currently available monitor (handheld or positioned on a cart) or a custom monitor for use with the soft tissue cutting device 400. Some embodiments may include multiple cameras 408 or other visualization components.

The pressure sensor 410 may be a pressure transducer, for example, or any other suitable pressure sensor. In some embodiments, the soft tissue cutting device 400 may include multiple pressure sensors 410. In alternative embodiments, the soft tissue cutting device 400 may include one or more other types of sensing components to sense one or more other parameters in the body. For example, the device 400 may include one or more thermocouples for sensing temperature. Using the embodiment shown, the soft tissue cutting device 400 may be used to sense pressure within the carpal tunnel before and/or after cutting the transverse carpal ligament. The sensed pressure may be converted to a digital signal and transmitted to a readout on the video monitor 412 on the handle 402 or to a separate monitor, thus helping the physician confirm that the ligament has been cut and pressure reduced within the carpal tunnel.

The pressure sensor 410 may be located at any suitable location on or along the device 100 for sensing pressure within the carpal tunnel. For example, the pressure sensor 410 may be located within the balloons, within the handle (e.g., in communication with fluid tubes in communication with the balloons), and/or at one or more other suitable locations for sensing pressure within the carpal tunnel.

In one method of performing a carpal tunnel release procedure, the shaft 404 of the soft tissue cutting device 400 may be inserted into the hand via an incision. As the distal end of the shaft 404 is advanced to a target location (and/or after advancing the distal end), the camera 408 may be used to confirm the location of the distal end relative to one or more anatomical structures (e.g., anatomical landmarks). Once the shaft 404 is in the desired location, the pressure sensor 410 may be used to sense pressure within the carpal tunnel. The blade 406 may then be activated and moved proximally along the shaft 404 to cut the transverse carpal ligament. The camera 408 may be used before, during, and/or after cutting, to visualize the surgical site and surrounding tissues. After the ligament is cut, the pressure sensor 410 may be used again to sense pressure in the area. When the physician is satisfied that the ligament has been cut and pressure reduced, the physician may then remove the shaft 404 from the hand.

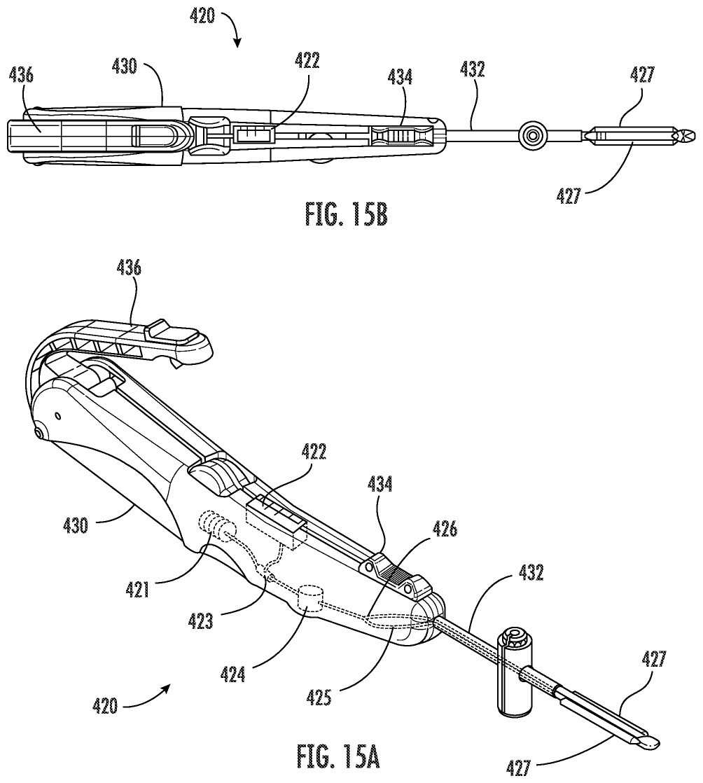

Referring now to FIGS. 15A and 15B (perspective and top views, respectively), another embodiment of a soft tissue cutting device 420 is illustrated. This embodiment of the soft tissue cutting device 420 includes many of the parts and features of embodiments described above, which will not be described again here in detail. For example, the soft tissue cutting device 420 includes a handle 430, a shaft 432 extending from the handle 430, a blade (not pictured) housed within and moveable along the shaft 432, two inflatable balloons 427 on either side of a distal portion of the shaft 432, a slider 434 on the handle 430 and coupled with the blade to move the blade back and forth along the shaft 432, and an inflation lever 436 coupled with the handle 430 for inflating the balloons 427. Again, these features will not be described in further detail here but may have any of the characteristics of analogous features in embodiments described above.

In the embodiment of FIGS. 15A and 15B, the soft tissue cutting device 420 further includes a bellows 421, tubing 425 leading from the balloons 427 to the bellows 421, a pressure gauge with indicator/display 422, a check valve 424, a T-connector 423, and a Y-connector 426. The balloons 427 are connected via tubing 425 to the Y-connector 426, which is connected via tubing 425 to the check valve 424, which is connected via tubing to the T-connector 423, which connects further tubing 425 to the pressure gauge with indicator/display 422 and the bellows 421. When the inflation lever 436 is depressed/squeezed by the user, the bellows 421 inflate the balloons 427, and pressure within the system is measured by the pressure gauge with indicator/display 422. This pressure measuring system may be used to confirm that the balloons 427 have inflated when priming the soft tissue cutting device 420 before performing a procedure. The system may also be used to measure pressure within the hand, or specifically within the carpal tunnel, before, during, and/or after cutting the transverse carpal ligament. For example, the user may take a first pressure measurement before cutting the transverse carpal ligament and a second pressure measurement after cutting the ligament.

The embodiments and features described above are illustrative in nature and are not meant to narrow the scope of the present disclosure. Any features or components described for one embodiment may be applied to any other embodiment.

1. A method of performing a carpal tunnel release procedure on a hand, the method comprising:
advancing a shaft of a soft tissue cutting device into the hand;
positioning a distal end of the shaft at or near a carpal tunnel;
using a visualization member on the distal end of the shaft to visualize at least one anatomical landmark;
moving a blade along the shaft to cut a transverse carpal ligament; and
removing the shaft of the soft tissue cutting device from the hand.

2. The method of claim 1, wherein the visualization member is selected from the group consisting of a camera, a light fiber, and an ultrasound transducer.

3. The method of claim 1, further comprising sensing pressure within the carpal tunnel using a pressure sensor in the soft tissue cutting device.

4. The method of claim 3, wherein sensing pressure comprises:
sensing a first pressure before cutting the transverse carpal ligament; and
sensing a second pressure after cutting the transverse carpal ligament.

5. The method of claim 4, further comprising:
converting the first pressure and the second pressure into digital numerical data; and
providing the digital numerical data on a display coupled with the soft tissue cutting device.

6. The method of claim 3, wherein sensing pressure comprises sensing pressure in at least one balloon coupled with the shaft of the soft tissue cutting device, and wherein the pressure sensor is located in a handle of the soft tissue cutting device.

7. The method of claim 1, further comprising inflating at least one balloon coupled with the shaft of the soft tissue cutting device to move one or more soft tissues away from the blade of the soft tissue cutting device.

8. A method of performing a carpal tunnel release procedure on a hand, the method comprising:
advancing a shaft of a soft tissue cutting device into the hand;
positioning a distal end of the shaft at or near a carpal tunnel;
sensing a first pressure in the hand, using a pressure sensor of the soft tissue cutting device;
moving a blade along the shaft to cut a transverse carpal ligament; and
sensing a second pressure in the hand, using the pressure sensor.

9. The method of claim 8, further comprising visualizing an area within the hand using a visualization member on the distal end of the shaft.

10. The method of claim 8, further comprising inflating at least one balloon coupled with the shaft of the soft tissue cutting device to move one or more soft tissues away from the blade of the soft tissue cutting device, wherein sensing the first pressure and the second pressure comprises sensing the first pressure and the second pressure in the at least one balloon, and wherein the pressure sensor is located in a handle of the soft tissue cutting device.

11. A device for performing a carpal tunnel release procedure on a hand, the device comprising:
a handle comprising a slider;
a shaft extending from the handle;
a blade on a distal portion of the shaft, coupled with the slider; and
a visualization member on the distal portion of the shaft.

12. The device of claim 11, wherein the visualization member is selected from the group consisting of a camera, a light fiber, and an ultrasound transducer.

13. The device of claim 11, further comprising at least one balloon coupled with the distal portion of the shaft and configured to move one or more soft tissues away from the blade.

14. The device of claim 13, further comprising a pressure sensor in the handle that senses pressure in the at least one balloon.

15. The device of claim 14 wherein the pressure sensor is selected from the group consisting of pressure transducers and thermocouples.

16. A device for performing a carpal tunnel release procedure on a hand, the device comprising:
a handle comprising a slider;
a shaft extending from the handle;
a blade on a distal portion of the shaft, coupled with the slider;
at least one balloon coupled with the shaft; and
a pressure sensor in the handle and coupled with the at least one balloon to sense pressure in the at least one balloon.

17. The device of claim 16, further comprising a visualization member coupled with the distal portion of the shaft, wherein the visualization member is selected from the group consisting of a camera, a light fiber, and an ultrasound transducer.

18. The device of claim 16, wherein the shaft comprises an upper surface with an opening, and wherein the blade moves out of the opening to cut tissue.

19. The device of claim 16, wherein the at least one balloon comprises two balloons disposed on two sides of the shaft, configured to move soft tissue away from the blade.
